(19) United States
(12) Patent Application Publication
(10) Pub. No.: US 2026/0241199 A1
(43) Pub. Date: Aug. 20, 2026

(54) WEARABLE PHOTOTHERAPY DEVICE

(71) Applicant: SHENZHEN KAIYAN MEDICAL EQUIPMENT CO., LTD, Shenzhen (CN)

(72) Inventor: ALAIN DIJKSTRA, Amstelveen (NL)

(73) Assignee: SHENZHEN KAIYAN MEDICAL EQUIPMENT CO., LTD, Shenzhen (CN)

(21) Appl. No.: 19/642,670

(22) Filed: Apr. 9, 2026

Related U.S. Application Data
(63) Continuation-in-part of application No. 18/295,861, filed on Apr. 5, 2023, now Pat. No. 12,629,538.

(51) Int. Cl. A61N 5/06; A61N 5/00

(52) U.S. Cl. CPC A61N 5/0616; A61N 2005/007; A61N 2005/0627; A61N 2005/0632; A61N 2005/0647; A61N 2005/0652; A61N 2005/0664

(57) ABSTRACT

A wearable phototherapy device comprises an outer layer, an inner layer, a plurality of light sources located between the outer layer and the inner layer, wherein the plurality of light sources is directed towards the inner layer, and a metallic layer provided on an inner surface of the inner layer, wherein the metallic layer is configured to conduct electrical current. The inner layer acts as a spacer between the metallic layer and the plurality of light sources. The device may be configured in various form factors including a face mask, patch, pad, mat, or wearable garment. A method of manufacturing the wearable therapy device is also provided.

### CROSS-REFERENCE TO RELATED APPLICATIONS

This application is a continuation-in-part of U.S. Application No. 18/295,861, titled Wearable Phototherapy Device, filed April 5, 2023, which is hereby incorporated by reference in its entirety.

### TECHNICAL FIELD

The present invention generally relates to wearable therapeutic devices. More specifically, the present invention relates to wearable devices configured to provide multiple therapeutic modalities, including phototherapy, electrical stimulation therapies, thermal therapy, and ultrasonic therapy.

### BACKGROUND ART

Light therapy devices have been known in the art for quite a while. Many such devices are designed to be wearable in nature such as in the shape of a face mask, an armband, or a belt for the waist region. Such devices in construction generally have an outer layer and a transparent inner layer and several LEDs located between the outer layer and the transparent inner layer. The LEDs have been located in such a manner that the LEDs are directed toward the inner transparent layer. In many such devices, the LEDs, in whole or in part, may be replaced with other light sources such as, but not limited to, halogen lamps or LASERs. In general, light sources, either LEDs or other light sources such as halogen lamps and LASERs, emit radiation between the infrared and ultraviolet ends of the electromagnetic spectrum. The aim of such devices is to impart irradiation to a specific body part of the user to cause skin regeneration and repair as the skin absorbs photons from the irradiation and enhances the production of Adenosine Tri-Phosphate (ATP) and collagen.

However such devices have the singular functionality of providing light therapy and are not able to combine other therapies, such as Transcutaneous Electrical Nerve Stimulation (TENS), Electronic Muscle Stimulation (EMS), and Radio-Frequency (RF) with light therapy. Phototherapy, however, alone may not be as effective, especially in the case of applications such as wound healing or pain relief. In such scenarios and also to enhance skin regeneration in general, a combination of therapies may be required to achieve optimal results. As a result, a user needs to purchase multiple devices each providing a different therapy. This adds to cost and complexity on the part of the user as purchasing multiple devices contributes to added costs and changing between the multiple devices contributes to the complexity of the operation.

Therefore, there is a need for a device that overcomes the disadvantages and limitations associated with the prior art and provides a more satisfactory solution.

### OBJECTS OF THE INVENTION

Some of the objects of the invention are as follows:

An object of the invention is to provide a wearable phototherapy device that includes a printed metallic layer that is electrically conductive.

Another object of the invention is to provide a wearable phototherapy device that also includes several metal electrodes interspersed along the metallic layer.

Another object of the invention is to provide a wearable phototherapy device that is capable of providing multiple therapies such as Transcutaneous Electrical Nerve Stimulation (TENS), Electronic Muscle Stimulation (EMS), Galvanic Therapy, Radio-Frequency (RF), etc.

Another object of the invention is to provide a wearable phototherapy device where the metallic layer has been provided in predetermined patterns.

Yet another object of the invention is to provide a wearable phototherapy device that that can take several forms such as a face mask, an armband, a belt, etc.

Another object of the invention is to provide a wearable phototherapy device wherein an inner layer acts as a spacer between a metallic layer and a plurality of light sources.

Another object of the invention is to provide a wearable phototherapy device with a spacing structure having spacer elements of different heights to accommodate the anatomical contours of a user.

Another object of the invention is to provide a wearable phototherapy device with distributed parallel conductive paths for uniform current distribution across light sources, metal electrode and plurality of regions of the metallic layer.

Another object of the invention is to provide a wearable phototherapy device with skin impedance sensing and adaptive current control.

Another object of the invention is to provide a wearable phototherapy device with artificial intelligence-based therapy selection and feedback loop functionality.

Another object of the invention is to provide a wearable phototherapy device capable of providing thermal therapy, including heating and cooling, and ultrasonic therapy.

Another object of the invention is to provide a wearable therapy device in various form factors, including patches, pads, mats, and wearable garments.

Yet another object of the invention is to provide a method of manufacturing a wearable therapy device.

### SUMMARY OF THE INVENTION

According to a first aspect of the present invention, there is provided a wearable phototherapy device, the wearable phototherapy device comprising an outer layer, an inner transparent silicone layer, a plurality of light sources located between the outer layer and the inner transparent silicone layer, wherein the plurality of light sources is directed towards the inner transparent silicone layer, and a metallic layer printed onto an inner surface of the inner transparent silicone layer, wherein the metallic layer is configured to conduct electrical current.

In one embodiment of the invention, the wearable phototherapy device further comprises a plurality of metal electrodes interspersed along the metallic layer.

In one embodiment of the invention, the plurality of metal electrodes are floating electrodes.

In one embodiment of the invention, the plurality of metal electrodes is configured to transmit electrical current within the metallic layer.

In one embodiment of the invention, a plurality of regions of the metallic layer between the interspersed plurality of metal electrodes correspond to a plurality of parts of the body of a user.

In one embodiment of the invention, the flow of current to the plurality of regions is configured to be individually controlled through control of the flow of current to individual metal electrodes of the plurality of metal electrodes.

In one embodiment of the invention, the plurality of metal electrodes is configured to emit Radio-Frequency (RF) waves to provide RF treatment to the body of a user.

In one embodiment of the invention, the plurality of metal electrodes are supported by elastic members at the points of attachment of the plurality of metal electrodes.

In one embodiment of the invention, the wearable phototherapy device is in the shape of a face mask.

In one embodiment of the invention, the metallic layer has been provided in the form of a pattern including a plurality of connected hexagons.

In one embodiment of the invention, the plurality of light sources includes Light Emitting Diodes (LEDs).

In one embodiment of the invention, the plurality of light sources is configured to emit radiation in visible light and infrared wavelengths of the electromagnetic spectrum.

In one embodiment of the invention, the metallic layer is configured to conduct electrical current to provide Galvanic therapy to a user, the Galvanic therapy including desincrustation and iontophoresis.

In one embodiment of the invention, the metallic layer is configured to conduct electrical current to provide Transcutaneous Electrical Nerve Stimulation (TENS) to a user.

In one embodiment of the invention, the metallic layer is configured to conduct electrical current to provide Electronic Muscle Stimulation (EMS) to a user.

In one embodiment of the invention, the wearable phototherapy device further comprises a battery, a control unit, and a mode controller.

In one embodiment of the invention, the control unit is configured to connect with an external communication device and receive an input from the external communication device.

In one embodiment of the invention, the inner transparent silicone layer includes a plurality of holes for the plurality of light sources and acts as a spacer between the metallic layer and the plurality of light sources.

In one embodiment of the invention, the plurality of metal electrodes are used to provide iontophoresis therapy.

In one embodiment of the invention, the plurality of metal electrodes are used to provide therapies including Electronic Muscle Stimulation (EMS), and Transcutaneous Electrical Nerve Stimulation (TENS).

In some embodiments, the metallic layer is divided into a plurality of conductive regions corresponding to different portions of the body of a user, such as the forehead, cheeks, chin, jawline, or region below the lips. The plurality of conductive regions may be electrically connected through multiple parallel or partially parallel conductive paths rather than a single conductive path. The conductive paths may be arranged such that substantially equal electrical path length and impedance are provided between a power source and each conductive region. In this manner, substantially uniform current and voltage may be delivered across the plurality of conductive regions, thereby reducing voltage drop along the metallic layer.

In some embodiments, electrical current is supplied to a conductive region from more than one direction. For example, the conductive paths may comprise mirrored conductive paths, looped conductive paths, segmented buses, mesh structures, or combinations thereof. Supplying current from multiple directions reduces local voltage drop and enables more uniform electrical stimulation and light output across the wearable device.

In some embodiments, the plurality of metal electrodes associated with the plurality of conductive regions are connected in parallel such that each region receives substantially the same current regardless of its distance from the power source. This arrangement is particularly advantageous in large-area or flexible wearable devices, such as facial masks, where a single-feed conductive path may otherwise result in reduced current, reduced light intensity, or non-uniform treatment in regions farther from the power source.

In some embodiments, the conductive paths are formed within or on the metallic layer using printed conductive traces, conductive meshes, laminated conductors, or embedded conductive structures. The conductive paths may be configured to preserve flexibility of the wearable device while maintaining substantially uniform electrical performance across the plurality of conductive regions.

In the context of the specification, the phrase “Transcutaneous Electrical Nerve Stimulation (TENS)” refers to electrical stimulation of muscles with low voltage electrical current using electrical electrodes for pain relief. The electrodes are placed at or near the nerves where the pain is located or at trigger points.

In the context of the specification, the phrase “Electronic Muscle Stimulation (EMS)” refers to electrical stimulation of specific muscle groups with a current of slightly greater magnitude than used in TENS to achieve muscle contractions. EMS can be used to enhance muscle strength, reduce swelling, relieve pain and help heal wounds.

In the context of the specification, the phrase “Galvanic Therapy” refers to application of direct current to the epidermis of a patient. The Galvanic Therapy aims to achieve a myriad of skin benefits like softening tissue, and softening blackheads by dilating the pores, stimulating cells, and driving ingredients deep into the epidermis. The Galvanic Therapy is of two types, desincrustation, and iontophoresis. Desincrustation includes the application of a desincrustation solution and then the movement of an active electrode to the area to be treated. Iontophoresis includes the application of galvanic current on the positive polarity. During an iontophoresis treatment, a product that has an acidic pH and is watersoluble can be used. Gels, serums, or even a mask can be applied all over the area to be treated. Moreover, an inactive (negative) electrode is placed on other parts of the body of the patient, such as under their shoulder, while a positive current is delivered into the skin via positively charged ions.

In the context of the specification, the phrase “Radio Frequency (RF) Treatment” refers to the application of RF waves to the dermis layer of a patient to generate heat, using a handheld probe. The handheld probe is applied topically on the upper layers of the skin as the RF waves penetrate deep into the skin, upto the dermis layer. The treatment aims to enhance the production of collagen and elastin for skin regeneration and growth.

In the context of the specification, the term “processor” refers to one or more of microprocessors, a microcontroller, a general-purpose processor, a Field Programmable Gate Array (FPGA) or an Application Specific Integrated Circuit (ASIC), and the like.

In the context of the specification, the phrase “storage memory” refers to one or more of a volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc., or a nonvolatile storage memory such as EPROM, EEPROM or flash memory or the like.

In the context of the specification, the phrase “communication interface” refers to a device or a module enabling direct connectivity via wires and connectors such as USB, HDMI, VGA, or wireless connectivity such as Bluetooth or Wi-Fi or Local Area Network (LAN) or Wide Area Network (WAN) implemented through TCP/IP, IEEE 802.x, GSM, CDMA, LTE or other equivalent protocols.

In the context of the specification, the term "historical" in execution of a command refers to anything pertaining to a time instant(s) that is earlier than a time instant of an initiation of the command.

In the context of the specification, the term, "real-time", refers to without intentional delay, given the processing limitations of hardware/software/firmware involved and the time required to accurately measure/receive/process/transmit data as practically possible.

In the context of this specification, terms like “light”, “radiation”, “irradiation”, “emission” and “illumination”, etc. refer to electromagnetic radiation in wavelength ranges varying from the visible light wavelengths (380-700 nm) to Infrared (IR) wavelengths (780 nm – 1 mm), wherein the range is inclusive of visible light and IR wavelengths. The IR radiation may also be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm).

In the context of the specification, a “polymer” is a material made up of long chains of organic molecules (having eight or more organic molecules) including, but not limited to, carbon, nitrogen, oxygen, and hydrogen as their constituent elements. The term polymer is envisaged to include both naturally occurring polymers such as wool, and synthetic polymers such as polyethylene and nylon.

In the context of the specification, “Light Emitting Diodes (LEDs)” are envisaged to be characterized by their superior power efficiencies, smaller sizes, rapidity in switching, physical robustness, and longevity when compared with incandescent or fluorescent lamps. In that regard, the plurality of LEDs may be through-hole type LEDs (generally used to produce electromagnetic radiations of red, green, yellow, blue and white colors), Surface Mount LEDs, Bi-color LEDs, Pulse Width Modulated RGB (Red-Green-Blue) LEDs, Organic LEDs (OLEDs) and high-power LEDs, etc.

Materials used in the one or more LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, and Boron, and Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used for generating a wider color gamut. White and other colored lightings may also be produced using phosphor coatings such as Yttrium Aluminum Garnet (YAG) in combination with a blue LED to generate white light and Magnesium doped potassium fluorosilicate in combination with blue LED to generate red light. Additionally, near Ultraviolet (UV) LEDs may be combined with europium-based phosphors to generate red and blue lights and copper and zinc doped zinc sulfide-based phosphor to generate green light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, discussion on generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the one or more LEDs may also be micro-LEDs described through U.S. Pat. Nos. 8,809,126 B2, 8,846,457 B2, 8,852,467 B2, 8,415,879 B2, 8,877,101 B2, 9,018,833 B2 and their respective family members, assigned to NthDegree Technologies Worldwide Inc., which are included herein by reference, in their entirety. The one or more LEDs, in that regard, may be provided as a printable composition of the micro-LEDs, printed on a substrate.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1 illustrates an exploded view of a wearable phototherapy device in accordance with an embodiment of the present invention;

FIG. 2 illustrates a rear perspective view of the wearable phototherapy device, in accordance with an embodiment

FIG. 3 illustrates a metallic layer with interspersed metal electrodes, in accordance with an embodiment of the present invention;

FIG. 4A illustrates a bottom perspective view of the wearable phototherapy device, in accordance with an embodiment of the present invention;

FIG. 4B illustrates a magnified view of a portion labeled as 4B in FIG. 4A;

FIG. 4C illustrates a magnified view of a portion labeled as 4C in FIG. 4A; and

FIG. 5 illustrates the wearable phototherapy device connected with an external communication device, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

It is envisaged that a wearable phototherapy device is provided that provides several alternative treatments to a user along with light therapy. Such alternative treatments may include Transcutaneous Electrical Nerve Stimulation (TENS), Electronic Muscle Stimulation (EMS), Radio-Frequency (RF), Galvanic therapy, etc. To that end, a metallic layer has been provided on an inner transparent layer of the device, in a manner that the metallic layer conducts electricity and makes contact with the skin of the user. The alternative treatments are achieved through the flow of electrical current in the metallic layer. To enhance the effectiveness of the metallic layer, several metal electrodes have been interspersed along the metallic layer. The metal electrodes form several regions that may be individually controlled through control of the flow of current to individual metal electrodes. Further, the wearable phototherapy device may take the shape of a face mask, an armband, a waist belt, etc. Referring to the drawings, the invention will now be explained in further detail.

While the following detailed description uses a face mask as an exemplary embodiment for purposes of illustration, it is to be understood that the present invention is not limited to face mask configurations. The structural elements, therapeutic functionalities, and manufacturing methods described herein are applicable to any wearable therapy device form factor, including but not limited to patches, pads, mats, armbands, leg wraps, back panels, neck wraps, hand wraps, foot wraps, body suits, shirts, pants, sleeves, gloves, socks, belts, and other wearable configurations. The face mask embodiment is presented as one non-limiting example, and a person of ordinary skill in the art would understand that the principles disclosed herein may be adapted and scaled for application to any body region or form factor without departing from the scope of the invention.

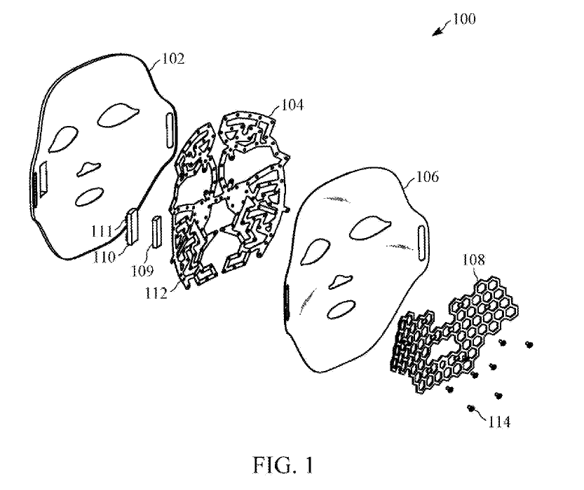

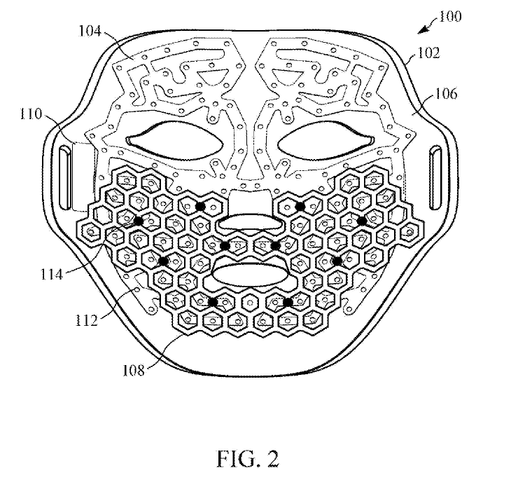

FIG. 1 illustrates an exploded view of a wearable phototherapy device 100 (hereinafter referred to as “the device 100”) in accordance with an embodiment of the present invention. FIG. 2 illustrates a rear perspective view of the device 100, in accordance with an embodiment. As illustrated in FIGS. 1 and 2, the device 100 is in the shape of a face mask. However, in several alternate embodiments, the device 100 may take several other forms, such as an armband, a jacket, or a waist belt. The device 100 includes an outer layer 102 and an inner transparent layer 106. In several embodiments of the invention, the inner transparent layer 106 is made up of silicone material, alternatively the inner transparent layer is made of Methyl Methacrylate Cross polymer silicone to shine the face more efficiently and enhances the light therapy effect. A plurality of light sources 112 is located between the outer layer 102 and the inner transparent layer 106. The plurality of light sources 112 have been provided on a flexible Printed Circuit Board (PCB) 104 and is directed towards the inner transparent layer 106. The inner transparent layer 106 further includes holes for fixing the plurality of light sources 112. Moreover, the inner transparent layer 106 creates a gap between the plurality of light sources 112 and a metallic layer 108. The gap will further illuminate the treatment area and diffuse the light from the plurality of light sources 112 to provide a better therapeutic effect. In several embodiments of the invention, the plurality of light sources 112 includes Light Emitting Diodes (LEDs). In several embodiments of the invention, the plurality of light sources 112 is configured to emit radiation in red and infrared frequencies of the electromagnetic spectrum.

Further, the metallic layer 108 has been printed onto an inner surface of the inner transparent layer 106. The metallic layer 108 is configured to conduct electrical current. In several embodiments of the invention, the metallic layer 108 has been provided in the form of a pattern including a plurality of connected hexagons. In several embodiments of the invention, the metallic layer 108 is configured to conduct electrical current to provide Transcutaneous Electrical Nerve Stimulation (TENS) to a user. In several alternate embodiments, the metallic layer 108 is configured to conduct electrical current to provide Electronic Muscle Stimulation (EMS) to a user. In several embodiments of the invention, the device 100 also includes a plurality of metal electrodes 114 interspersed along the metallic layer 108.

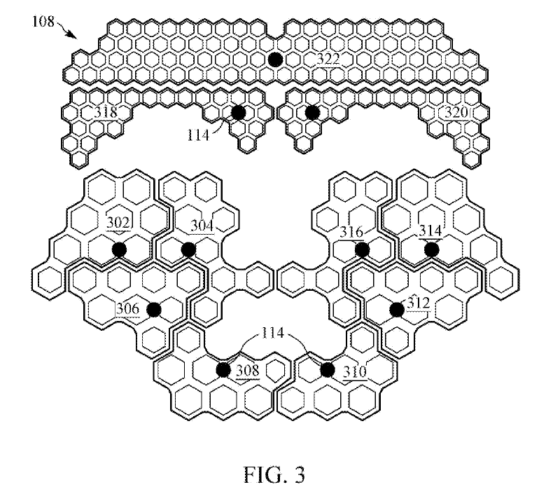

FIG. 3 illustrates the metallic layer 108 with the interspersed plurality of metal electrodes 114, in accordance with an embodiment of the present invention. The plurality of metal electrodes 114 is configured to transmit electrical current within the metallic layer 108. The plurality of metal electrodes 114 divides the metallic layer 108 into a plurality of regions 302, 304, 306, 308, 310, 312, 314, 316, 318, 320 and 322. The plurality of regions 302, 304, 306, 308, 310, 312, 314, 316, 318, 320 and 322 may then correspond to a plurality of parts of the body of a user. For example, if the device 100 is in shape of a face mask, then one region 304 may correspond to upper jaw region, another region 302 may correspond to area surrounding the eye and yet another region 306 may correspond to cheek of the user. In this manner, the whole face of the user may receive electrical stimulation from the plurality of metal electrodes 114 and the metallic layer 108. Also, the plurality of regions 302, 304, 306, 308, 310, 312, 314, 316, 318, 320 and 322 are disconnected from each other.

In that manner, the flow of current to the plurality of regions 302, 304, 306, 308, 310, 312, 314, 316, 318, 320 and 322 may be configured to be individually controlled through control of the flow of current to individual metal electrodes of the plurality of metal electrodes 114. In that regard, the flow of current to the area surrounding the eyes, the upper jaw, and the cheek of the user may be controlled through control of the flow of current to the individual electrodes, thereby providing an option for directed and selective treatment of specific part of the body of the user. The application of TENS, EMS and Galvanic Therapy also may be directed to specific part of the body of the user. Another advantage of the plurality of metal electrodes 114 is that they can be used to provide Radio Frequency (RF) treatment. Through electromagnetic interactions, the plurality of metal electrodes 114 may be configured to emit Radio-Frequency (RF) waves to provide RF treatment to the body of a user. In one embodiment of the invention, the plurality of metal electrodes 114 are also configured to balance the pH level of the contact area through electrolysis. In one embodiment of the invention, the plurality of metal electrodes 114 are floating electrodes. In several embodiments of the invention, the metallic layer 108 and the plurality of metal electrodes 114 are configured to conduct electrical current to provide Galvanic therapy to a user, the Galvanic therapy including desincrustation and iontophoresis.

Desincrustation involves the application of an alkaline solution with negatively charged ions to an affected area. The alkaline desincrustation solution is applied to both the skin and the gauze or cotton covering the active electrode, which traditionally is a prong or a tweezer, this is then moved over the affected area. In the current embodiment, the plurality of metal electrodes 114 and the metallic layer 108 act as the active electrodes by connecting them with a negative terminal of a power source such as a battery. For example, when the device 100 is in the form of a face mask, the desincrustation solution is applied to the skin of the face of a user and then the device 100 is applied to the face of the user. The negative ions in the solution are repelled by the plurality of metal electrodes 114 and the metallic layer 108, causing an alkaline reaction in the skin. This works on the theory that like poles repel and opposite poles attract. The alkaline desincrustation solution, combined with the action of the active negative electrode, results in the “saponification” of sebum. This alkali and sebum reaction forms sodium hydroxide (lye) due to the fatty stearic acids in sebum, reacting with the alkali to form soap. In the skin, the reaction softens and liquefies sebum, and this facilitates the easier release of blackheads.

Iontophoresis involves active transdermal drug delivery involving delivery of drug ions through the skin using low level electric current. When Direct Current (DC) is applied to an ionized drug solution, ions that have same charge as the current are repelled by the current and delivered through the skin. For example, the ionized drug solution may be applied to the face of the user, and the device 100 is embodied as an facemask. The DC current is applied through the metallic layer 108 and the plurality of metal electrodes 114. The ions in the drug solution are repelled and absorbed through the face of the user and provide skin enhancement effect to the user. Iontophoresis can be used with water soluble ionic medications to treat acute tendonitis, pain associated with calcific deposits, and provide dermal anesthesia. Iontophoresis has several advantages over alternatives of injections and oral medications. Iontophoresis is virtually painless and is non-invasive minimizing the risk of infection and tissue necrosis and tendon rupture.

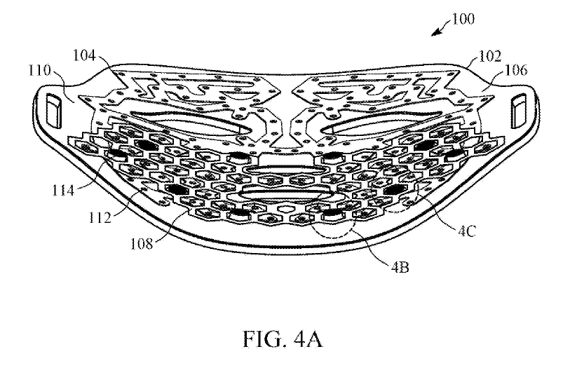

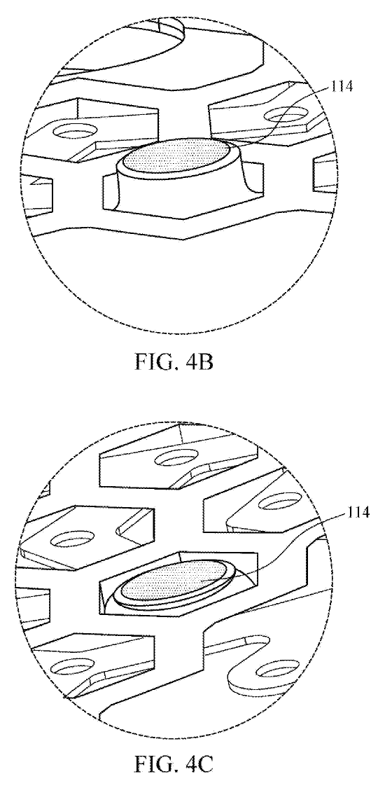

FIG. 4A illustrates a bottom perspective view of the device 100. The bottom perspective view illustrates the outer layer 102, the flexible circuit board 104, the inner transparent layer 106, the metallic layer 108, the control unit 110, the plurality of light sources 112, and the plurality of metal electrodes 114 as described in the aforementioned description. It is further envisaged that the plurality of metal electrodes 114 are floating electrodes. In that regard, the plurality of metal electrodes 114 adapt to the shape of a portion of the body on which the device 100 has been worn.

FIG. 4B illustrates a magnified view of a portion of the device 100 labeled as 4B in FIG. 4A. FIG. 4B illustrate the magnified view of the device 100, when the device 100 is not in use. The plurality of metal electrodes 114 as floating electrodes in that manner may be supported by elastic members at the points of attachment of the plurality of metal electrodes 114. In several embodiments of the invention, the elastic members may be made up of silicon. The elastic members allow the plurality of metal electrodes 114 to protrude upwards to a height greater than the height of the metallic layer 108. FIG. 4C illustrates a magnified view of a portion of the device 100 labeled as 4C in FIG. 4A. FIG. 4C illustrates the magnified view of the device 100, when the device 100 is in use. In FIG. 4C, the device 100 is making contact with the skin of the user, and the elastic members have been compressed to the height of metallic layer 108. Due to the elastic members and adaptability with the shape of the portion of the body, the plurality of metal electrodes 114 as floating electrodes provide relatively more comfortable effect on the portion of the body of the user. Moreover, the elastic members ensure that there is no direct contact between the plurality of light sources 112 and the skin of the user. For example, if the device 100 has been configured as a face mask, then the plurality of metal electrodes 114 as floating electrodes will provide relatively more comfortable effect on the face of the user and the elastic members and the transparent layer 106 will prevent direct contact of the plurality of light sources 112 and the facial skin of the user. This would prevent overheating and/or burning of the facial skin of the user.

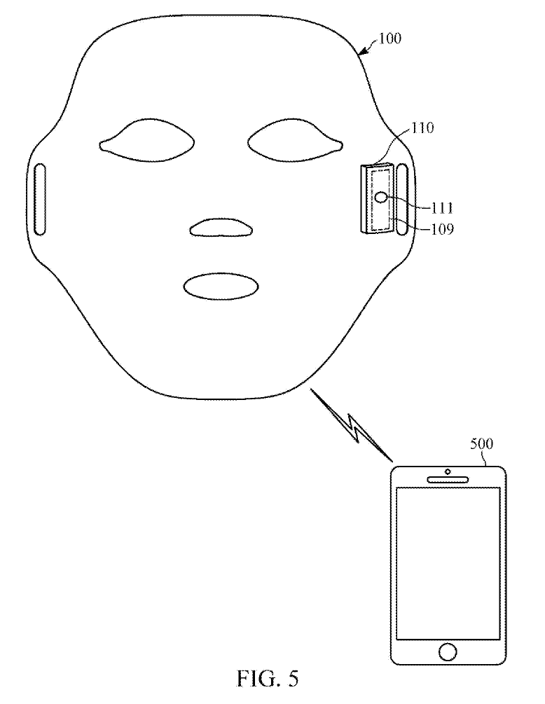

FIG. 5 illustrates the device 100 connected with an external communication device 500, in accordance with an embodiment of the present invention. The device 100 includes a battery 109, a control unit 110, and a mode controller 111. The battery 109 may be a rechargeable battery, such as Nickel-Metal-Hydride, Lithium-ion or Lithium-polymer. The mode controller 111 may allow switching between several different therapies, such as Galvanic Therapy, TENS, EMS, and RF therapy. The control unit 110 includes a processor, a memory unit and a communication interface. The memory unit includes machine-readable instructions for the processor to execute. The communication with the external communication device 500 is routed through the communication interface. The control unit 110 is configured to connect with the external communication device 500 and receive an input. The input may correspond to switching between different therapies, such as TENS, EMS, RF and Galvanic Therapy, switching between continuous and pulse mode of operation of the LEDs or modification of emission characteristics of the plurality of LEDs such as wavelength of the irradiation.

While the inner transparent layer 106 is described in several embodiments as being made of silicone material, it is to be understood that the inner layer may be made of various other materials without departing from the scope of the invention. The inner layer may comprise any optically transparent or translucent flexible material suitable for skin contact, including but not limited to silicone, thermoplastic polyurethane (TPU), thermoplastic elastomer (TPE), polyethylene, polypropylene, polyurethane, hydrogel, or other biocompatible polymeric materials. The inner layer may also comprise composite materials or multi-layer structures combining different materials to achieve desired optical, thermal, mechanical, or biocompatibility properties. The selection of material for the inner layer may depend on the specific application, form factor, and therapeutic requirements of the device 100.

In several embodiments, the device 100 includes a spacing structure comprising a plurality of spacer elements corresponding to a plurality of conductive regions of the metallic layer 108. The spacer elements may have different heights in different regions of the device 100. The different heights of the spacer elements may be configured to accommodate the anatomical contours of a user and to control spacing between the metallic layer 108 and the user across different regions. For example, when the device 100 is configured as a face mask, the spacer elements in regions corresponding to the nose, cheekbones, and forehead may have different heights to conform to the varying contours of the face. The inner transparent silicone layer 106 may function as the spacing structure, with varying thickness across different regions to provide the variable spacing. In some embodiments, the spacer elements may be integrally formed with the inner transparent silicone layer 106. In other embodiments, the spacer elements may be separate components attached to or embedded within the inner transparent silicone layer 106.

In various embodiments, the metallic layer 108 or conductive layer may be disposed within, on, or integrated with a flexible substrate using various methods. The conductive layer may be printed onto the flexible substrate using conductive inks or pastes. Alternatively, the conductive layer may be embedded within the flexible substrate during manufacturing. In some embodiments, the conductive layer may be laminated onto the flexible substrate as a separate conductive film or foil. The conductive layer may also be coated onto the flexible substrate through processes such as sputtering, vapor deposition, or electroplating. In further embodiments, the conductive layer may be insert-molded with the flexible substrate, wherein the conductive material is placed in a mold, and the flexible substrate material is molded around it. The flexible substrate may comprise silicone, thermoplastic polyurethane, or other biocompatible flexible materials.

In several embodiments, the plurality of metal electrodes 114 may protrude or stud out through the inner transparent silicone layer 106 to make direct contact with the skin of the user. In such embodiments, the device 100 may operate without the metallic layer 108, with the plurality of metal electrodes 114 providing the electrical stimulation directly to the skin. The inner transparent silicone layer 106 may include a plurality of holes or apertures through which the plurality of metal electrodes 114 extend. In some embodiments, the plurality of metal electrodes 114 may be detachable from the device 100. The detachable functionality allows for replacement of worn or damaged electrodes, cleaning and sterilization of the electrodes, and customization of electrode configurations for different treatment protocols. The detachable electrodes may be secured to the device 100 through snap-fit connections, magnetic attachments, threaded connections, or other releasable fastening mechanisms.

In several embodiments, the device 100 may be configured to provide electrical stimulation therapies without the plurality of light sources 112. In such embodiments, the inner transparent silicone layer 106 acts as a spacer between the metallic layer 108 and the flexible PCB 104. The flexible PCB 104 may include electronic circuitry for controlling electrical stimulation therapies such as microcurrent therapy, Transcutaneous Electrical Nerve Stimulation (TENS), Electronic Muscle Stimulation (EMS), and Galvanic therapy. In such embodiments, the metallic layer 108 and the plurality of metal electrodes 114 may serve as microcurrent electrodes for delivering electrical stimulation to the skin of the user. The spacing provided by the inner transparent silicone layer 106 maintains separation between conductive elements on the flexible PCB 104 and the metallic layer 108 to prevent electrical interference and ensure proper current delivery to the skin. The device 100 in such embodiments may be configured as a therapy device providing electrical stimulation therapies without phototherapy functionality.

In several embodiments, the control unit 110 includes skin impedance sensing circuitry configured to measure the electrical impedance of the skin of the user. The skin impedance measurements may be used to assess skin hydration levels, ensure proper electrode contact, and adjust therapy parameters accordingly. The control unit 110 may include adaptive current control configured to automatically adjust the electrical current delivered to the metallic layer 108 and the plurality of metal electrodes 114 based on the measured skin impedance and other parameters. The control unit 110 may be configured to provide zone-specific therapy control, wherein the flow of current to individual regions of the metallic layer 108 corresponding to different body zones may be independently controlled based on the skin impedance measurements for each zone. The zone-specific therapy control may enable customized treatment intensity, duration, and therapy type for different body regions based on the specific skin conditions and therapeutic requirements of each zone. In some embodiments, the control unit 110 includes artificial intelligence (AI) based therapy selection configured to analyze user data, treatment history, and skin condition to recommend or automatically select optimal therapy protocols. The AI-based therapy selection may utilize machine learning algorithms trained on treatment outcome data. The control unit 110 may be configured to connect with a mobile application on the external communication device 500 for app-based personalization, allowing the user to customize treatment parameters, track treatment progress, and receive personalized recommendations. The control unit 110 may further include feedback loop functionality incorporating temperature sensing and hydration sensing to monitor skin conditions during treatment and adjust therapy parameters in real-time to optimize treatment efficacy and safety.

In several embodiments, the device 100 is configured to provide thermal therapy in addition to phototherapy and electrical stimulation therapies. The device 100 may include one or more heating elements configured to provide heat therapy to the skin of the user. The heating elements may comprise resistive heating elements, Peltier devices, or other thermoelectric elements. Heat therapy may enhance blood circulation, relax muscles, and improve the absorption of topical treatments. In some embodiments, the device 100 includes one or more cooling elements configured to provide cooling therapy to the skin of the user. The cooling elements may comprise Peltier devices, thermoelectric coolers, or channels for circulating a cooling fluid. Cooling therapy may reduce inflammation, soothe irritated skin, and provide a refreshing sensation. The control unit 110 may be configured to control the heating and cooling elements to provide alternating hot and cold therapy or to maintain a target temperature at the skin interface. In an embodiment, the metallic layer is configured to provide the function of the heating element and the cooling element.

In several embodiments, the device 100 includes one or more ultrasonic transducers configured to provide ultrasonic therapy to the skin of the user. The ultrasonic transducers may be positioned between the outer layer 102 and the inner transparent silicone layer 106, or may be integrated with the flexible PCB 104. The ultrasonic transducers may be configured to emit ultrasonic waves at frequencies ranging from about 1 MHz to about 3 MHz for therapeutic applications. Ultrasonic therapy may enhance the penetration of topical treatments, stimulate collagen production, improve blood circulation, and provide deep tissue massage effects. The control unit 110 may be configured to control the frequency, intensity, and duration of the ultrasonic therapy. In some embodiments, the ultrasonic therapy may be combined with phototherapy and electrical stimulation therapies to provide a comprehensive multimodal treatment.

In several embodiments, the device 100 may be configured as a patch, a pad, or a mat for localized treatment of specific body areas. The patch configuration may be smaller than the face mask configuration and may be applied to areas such as the neck, hands, joints, or specific treatment sites. The patch may include an adhesive layer for securing the patch to the skin of the user. The pad or mat configuration may be used for the treatment of larger flat body areas such as the back, abdomen, or limbs. In some embodiments, the device 100 may be configured as a wearable garment such as a shirt, pants, sleeve, glove, sock, or body suit. The wearable garment configuration may incorporate the plurality of light sources 112, the metallic layer 108, and the plurality of metal electrodes 114 distributed across the garment to provide therapy to larger body areas or multiple body regions simultaneously. The wearable garment may be made of flexible, breathable materials and may include the control unit 110 in a pocket or attached module.

The structural elements of the device 100 may be adapted for different form factors and body regions. For an armband or leg wrap configuration, the outer layer 102, the flexible PCB 104, the inner transparent silicone layer 106, and the metallic layer 108 may be formed in an elongated shape configured to wrap around a limb. For a back panel or mat configuration, the layers may be formed in a larger, substantially flat shape. For a neck wrap configuration, the layers may be formed in a curved shape configured to conform to the contours of the neck. The plurality of regions of the metallic layer 108 may be configured to correspond to different muscle groups or treatment zones depending on the form factor. The spacing structure and spacer elements may be configured with heights appropriate for the anatomical contours of the specific body region. The control unit 110 may be positioned at different locations depending on the form factor, such as at an end of an armband, at a corner of a back panel, or integrated into a strap or attachment mechanism.

In several embodiments, the emitter array comprising the plurality of light sources 112 and a plurality of metal electrodes 114 are supplied by multiple electrically parallel or partially parallel conductive paths rather than a single serial feed, distributing drive current across the array and electrodes. The conductive paths may be arranged to equalize effective electrical path length and impedance to groups of emitters and electrodes, reducing local voltage drop. The architecture may include segmented buses, mirrored feeds, or looped interconnects that deliver power from multiple directions to each emitter region. Current sharing across multiple paths may reduce peak current density in any single conductor, maintaining uniform drive conditions while allowing thin, flexible conductors suitable for conformable devices.

In an embodiment of the present invention, a method of manufacturing the device 100 or a wearable therapy device is provided. The method comprises forming a flexible substrate. The flexible substrate may be formed by molding, casting, or extruding a flexible material such as silicone or thermoplastic polyurethane. The method may further comprise integrating a conductive layer within or on the flexible substrate. Integrating the conductive layer may include printing, embedding, laminating, coating, or insert-molding the conductive layer as described herein. The method may further comprise positioning a plurality of light sources relative to the conductive layer. The plurality of light sources may be positioned on a flexible PCB and aligned with holes or apertures in the flexible substrate. The method further comprises forming a spacing structure between the conductive layer and the plurality of light sources. The spacing structure is formed integrally with the flexible substrate or as a separate component. In some embodiments, forming the spacing structure includes forming spacer elements of different heights in different regions of the device to accommodate the anatomical contours of a user.

The method of manufacturing the device 100 further comprises assembling the layers of the device 100. Assembling the layers includes bonding or adhering the outer layer 102 to the flexible PCB 104 using an adhesive layer or thermal bonding. The inner transparent silicone layer 106 is attached to the flexible PCB 104 by aligning the plurality of holes in the inner transparent silicone layer 106 with corresponding positions of the plurality of light sources 112 on the flexible PCB 104, such that the plurality of light sources 112 are received within or extend through the plurality of holes, and bonding the inner transparent silicone layer 106 to the flexible PCB 104. In some embodiments, the layers may be laminated together using heat and pressure. The assembly process may further include sealing or encapsulating the device 100 to protect the electronic components from moisture and environmental contaminants.

The method further comprises integrating the plurality of metal electrodes 114 with the device 100. Integrating the plurality of metal electrodes 114 includes positioning the plurality of metal electrodes 114 at predetermined locations along the metallic layer 108. The plurality of metal electrodes 114 is electrically connected to the detachable metallic layer 108 through soldering, conductive adhesive, or mechanical contact. In embodiments where the plurality of metal electrodes 114 is detachable, the method may include installing snap-fit receptacles, magnetic attachment points, or threaded inserts at the electrode locations. In embodiments where the plurality of metal electrodes 114 are floating electrodes, the method may include attaching elastic members to the plurality of metal electrodes 114 at the points of attachment to allow the electrodes to protrude and compress during use.

The method further comprises mounting the control unit 110 to the device 100. Mounting the control unit 110 includes positioning the control unit 110 on the outer layer 102 or in a housing attached to the device 100. The method further includes establishing electrical connections between the control unit 110 and the flexible PCB 104, the metallic layer 108, and the plurality of metal electrodes 114. The electrical connections are made through flexible ribbon cables, conductive traces, or wire harnesses. The method further includes installing the battery 109 and the mode controller 111 in proximity to the control unit 110.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to be providing the broadest scope consistent with the principles and the novel and inventive features disclosed or suggested herein. Accordingly, the invention is anticipated to hold on to all other such alternatives, modifications, and variations that fall within the scope of the present invention and appended claims.

1. A wearable phototherapy device, the wearable phototherapy device comprising:
an outer layer;
an inner layer;
a plurality of light sources located between the outer layer and the inner layer, wherein the plurality of light sources is directed towards the inner layer; and
a metallic layer provided on an inner surface of the inner layer, wherein the metallic layer is configured to conduct electrical current;
wherein the inner layer acts as a spacer between the metallic layer and the plurality of light sources.

2. The wearable phototherapy device of claim 1, wherein the inner layer includes a plurality of holes for the plurality of light sources.

3. The wearable phototherapy device of claim 1, further comprising a plurality of metal electrodes interspersed along the metallic layer.

4. The wearable phototherapy device as claimed in claim 3, wherein the plurality of metal electrodes is configured to transmit electrical current within the metallic layer and wherein the flow of current to the plurality of regions is configured to be individually controlled through control of the flow of current to individual metal electrodes of the plurality of metal electrodes.

5. The wearable phototherapy device of claim 3, wherein the plurality of metal electrodes are floating electrodes supported by elastic members at points of attachment of the plurality of metal electrodes, and wherein the plurality of metal electrodes are detachable from the wearable phototherapy device through one of snap-fit connections, magnetic attachments, or threaded connections.

6. The wearable phototherapy device of claim 3, wherein the plurality of metal electrodes correspond to a plurality of conductive regions of the metallic layer, and wherein the plurality of conductive regions are electrically connected through multiple parallel or partially parallel conductive paths configured to provide substantially equal electrical path length and impedance between a power source and each conductive region, such that substantially uniform current and voltage are delivered to the plurality of conductive regions and voltage drop across the metallic layer is reduced.

7. The wearable phototherapy device of claim 1, wherein the inner layer comprises a spacing structure having a plurality of spacer elements corresponding to a plurality of conductive regions of the metallic layer, wherein the spacer elements have different heights in different regions of the wearable phototherapy device to accommodate anatomical contours of a user.

8. The wearable phototherapy device of claim 1, further comprising a control unit including skin impedance sensing circuitry configured to measure electrical impedance of skin of a user, wherein the control unit includes adaptive current control configured to automatically adjust electrical current delivered to the metallic layer based on the measured electrical impedance.

9. The wearable phototherapy device of claim 1, further comprising a control unit including artificial intelligence based therapy selection configured to analyze user data and skin condition to select therapy protocols, and feedback loop functionality incorporating temperature sensing and hydration sensing to adjust therapy parameters in real-time.

10. The wearable phototherapy device of claim 1, wherein the metallic layer is configured to conduct electrical current to provide Galvanic therapy to a user, the Galvanic therapy including desincrustation and iontophoresis, and wherein the metallic layer is provided in a form of a pattern including a plurality of connected hexagons

11. A method of manufacturing a wearable therapy device comprising:
forming a flexible substrate by one of molding, casting, or extruding a biocompatible flexible material;
integrating a conductive layer within or on the flexible substrate, wherein the conductive layer is configured to conduct electrical current for delivering therapy to skin of a user;
mounting a plurality of light sources on a flexible printed circuit board;
positioning the flexible printed circuit board relative to the conductive layer such that the plurality of light sources is directed toward the conductive layer; and
forming a spacing structure between the conductive layer and the plurality of light sources, wherein the spacing structure maintains a gap between the conductive layer and the plurality of light sources.

12. The method of claim 11, wherein integrating the conductive layer comprises one of printing the conductive layer onto the flexible substrate using conductive inks or pastes, embedding the conductive layer within the flexible substrate during molding, laminating a conductive film or foil onto the flexible substrate, coating the conductive layer onto the flexible substrate through sputtering, vapor deposition, or electroplating, or insert-molding the conductive layer by placing conductive material in a mold and molding the flexible substrate around the conductive material.

13. The method of claim 11, wherein forming the spacing structure comprises forming a plurality of spacer elements having different heights in different regions of the wearable therapy device, wherein the different heights are configured to accommodate anatomical contours of a user.

14. The method of claim 11, further comprising assembling layers of the wearable therapy device by bonding or adhering an outer layer to the flexible printed circuit board, attaching the flexible substrate to the flexible printed circuit board by aligning a plurality of holes in the flexible substrate with the plurality of light sources, and sealing or encapsulating the wearable therapy device to protect electronic components from moisture and environmental contaminants.

15. The method of claim 11, further comprising integrating a plurality of metal electrodes with the wearable therapy device by positioning the plurality of metal electrodes at predetermined locations along the conductive layer, and electrically connecting the plurality of metal electrodes to the conductive layer, wherein the plurality of metal electrodes are detachable from the wearable therapy device through snap-fit receptacle, magnetic attachment point, or threaded insert at the electrode location.

16. The method of claim 11, further comprising attaching elastic members to the plurality of metal electrodes at points of attachment to allow the plurality of metal electrodes to protrude and compress during use as floating electrodes.

17. A therapy device, comprising:
an outer layer;
an inner transparent silicone layer;
a circuit board located between the outer layer and the inner transparent silicone layer, wherein the circuit board is configured to provide electrical connection to electronic circuitry; and
a metallic layer provided on an inner surface of the inner transparent silicone layer, wherein the metallic layer is configured to conduct electrical current;
wherein the inner transparent silicone layer acts as a spacer between the metallic layer and the circuit board.

18. The therapy device of claim 17, further comprising a plurality of light sources located on the circuit board, wherein the plurality of light sources is configured to provide light therapy to skin of a user.

19. The therapy device of claim 17, further comprising one or more thermal elements configured to provide heat therapy to skin of a user and one or more cooling elements configured to provide cooling therapy to skin of a user.

20. The therapy device of claim 17, further comprising one or more ultrasonic transducers configured to provide ultrasonic therapy to skin of a user, wherein the therapy device is configured as one of a patch, a pad, or a mat for localized treatment of specific body areas.
